(19) United States
(12) Patent Application Publication
(10) Pub. No.: US 2020/0323921 A1
(43) Pub. Date: Oct. 15, 2020

(54) HUMAN MILK OLIGOSACCHARIDES AND SYNTHETIC COMPOSITIONS THEREOF FOR MICROBIOTA MODULATION

(71) Applicant: GLYCOM A/S, Hørsholm (DK)

(72) Inventors: Louise Kristine VIGSNÆS, Copenhagen NV (DK); Bruce MCCONNELL, La Tour De Peilz (CH)

(21) Appl. No.: 16/768,414

(22) PCT Filed: Nov. 30, 2018
(86) PCT No.: PCT/IB2018/059501

§ 371 Date: May 29, 2020

(30) Foreign Application Priority Data
Nov. 30, 2017 (DK) PA 2017 00680
Dec. 5, 2017 (DK) PA 2017 00690

(51) Int. Cl. A61K 35/20; A61K 9/00; A61P 1/00

(52) U.S. Cl. CPC A61K 35/20; A61P 1/00; A61K 9/0053

(57) ABSTRACT

The present invention relates to methods, compounds and compositions for modulating the microbiota in the gastro-intestinal tracts of humans, particularly for increasing the abundance of Akkermansia in the gut microbiota of humans.

### FIELD OF THE INVENTION

This invention relates to methods, compounds and compositions for modulating the microbiota in the gastro-intestinal tracts of humans, particularly for increasing the abundance of Akkermansia in the gut microbiota of humans.

### BACKGROUND OF THE INVENTION

It has been estimated that the human intestine harbours 10¹³ to 10¹⁴ bacterial cells and the number of bacteria outnumbers the total number of cells in the body by a factor of 10. The microbiota of the human intestine is a complex and very dynamic microbial ecosystem, which serves numerous important functions for its human host, including protection against pathogens, induction of immune regulatory functions, nutrient processing and metabolic functions. The intestinal microbiota consists of various populations, which are important to preserve human health, and recent research has been able to link imbalances in the intestinal bacterial population to both intestinal and extra-intestinal inflammatory diseases.

The intestinal mucus is composed of two layers; an inner layer devoid of bacteria and a thicker outer layer, which is colonized and degraded by commensal bacteria. The colonic mucus constitutes a complex fluid, which is rich in gel-forming mucins. The mucins are large glycoproteins characterized by abundant and variable O-linked glycans attached to hydroxy amino acids clusters. Only some specific intestinal bacteria (the mucus-colonizing bacteria) are able to use mucins as a nutrient source because they possess the enzymatic machinery necessary for the breakdown of the mucin glycans. The mucus-colonizing bacteria can protect the host against intestinal pathogens, contribute to restoration of the microbiota and impact host responses. Akkermansia (incl. the type bacterial species Akk. muciniphila) is one of the most abundant mucus-colonizing bacteria in the human intestinal microbiota and is one of the handful of core microbes identified in humans. Akkermansia is capable of utilizing mucus as a sole carbon and nitrogen source due to the presence of genes coding for the enzymes; sialidases, fucosidases, N-acetyl-β-glucosaminidases and GlcNAc-sulfatase. Biochemical analysis of the mucin-degrading enzymes of Rumminococcus gnavus (another mucus-colonizing bacterium associated with inflammatory bowel disease) and Akkermansia has revealed marked differences in their phenotype. For R. gnavus, a selfish phenotype was revealed, while Akkermansia was compatible with a phenotype that stimulated tropic chains. This suggests that Akkermansia is involved in driving the existence of the mucosal community.

Several studies have reported a reduction in the abundance of Akkermansia in various disorders and diseases in humans. The majority of these include intestinal diseases such as inflammatory bowel disease, but also extra-intestinal, such as atopy, obesity, type 2 diabetes, hypertension, liver diseases and autism. This indicates that Akkermansia is associated with a protective and/or anti-inflammatory role, which is lost in the aforementioned diseases.

Several preclinical studies have shown that Akk. muciniphila can counteract the deleterious metabolic features of a high-fat diet in rodents including restoring epithelial integrity (mucus thickness), decreasing metabolic endotoxemia (serum LPS) and ameliorating metabolic profiles such as glucose tolerance. This indicates improvement in metabolic symptoms associated with obesity, diabetes and liver diseases. It has also been shown that Akk. muciniphila can increase the number of regulatory T cells and goblet cells in the intestine, leading to immune signalling and mucus production. Furthermore, Akk. muciniphila has been studied for its ability to affect nucleotide oligomerization domain like receptors and Toll-Like Receptors (TLRs). These receptors are a special group of membrane and intracellular proteins that play a critical role in immune regulation and are directly involved in the recognition of bacterial constituents by the immune system. A study has showed that Akk. muciniphila specifically interact with TLR2. This receptor is important in modulating intestinal homeostasis and host metabolism, and dysfunction of this has been associated with diseases such as non-coeliac wheat sensitivity, IBD and IBS. This indicates that Akkermansia is actively communicating with the host both by stimulating mucin production (e.g. depending on mucus thickness) and by inducing regulatory immune responses.

Selective stimulation of specific intestinal bacteria to promote their growth and metabolic activity (e.g. production of SCFA) could be a helpful approach in creating a beneficial intestinal microbial community that it able to regulate immune and metabolic functions. For example, a study has described that consuming fructooligosaccharides increased the Akkermansia population and improved host health. However, some individuals are sensitive to fructooligosaccharides, and side effects such as bloating, abdominal pain and increased flatulence occur.

It has also been described (WO 2014/076246) that repeated administration of Akkermansia impacts some underlying dysfunctions associated with obesity and related disorders, i.e. metabolic dysfunctions, low grade inflammatory state associated to higher blood lipopolysaccharides (LPS) levels and impaired gut barrier function. Therefore, administering Akkermansia as a probiotic supplementation could be an approach. However, no Akkermansia probiotic has been approved for food use and the addition of an exogenous organism may not be sufficient to fully promote a beneficial effect.

Human milk oligosaccharides (HMOs) are a heterogeneous mixture of soluble glycans found in human milk. They are the third most abundant solid component after lactose and lipids in human milk and are present in concentrations of 5-25 g/l (Bode: Human milk oligosaccharides and their beneficial effects, in: Handbook of dietary and nutritional aspects of human breast milk (Zibadi et al., eds.), pp. 515-31, Wageningen Academic Publishers (2013)). The structure of the HMOs is similar to the 0-glycans found in mucus and the N-glycans found on human cells. HMOs are resistant to enzymatic hydrolysis in the small intestine and are thus largely undigested and unabsorbed. The majority of HMOs that reach the colon serve as substrates to shape the gut ecosystem by selectively stimulating the growth of specific bacteria. HMOs are believed to substantially modulate the infant gut microbiota and play a decisive role in the differences in the microbiota of formula-fed and breast-fed infants. However, it is not known if HMOs can impact the adult microbiota community stimulating the growth of Akkermansia.

Metabolic end products such as short chain fatty acids (acetate, propionate and butyrate), produced during carbohydrate fermentation, also contribute to intestinal functionality and beneficial attributes of Akkermansia. Akkermansia produces acetate and propionate as a results of mucin degradation. These metabolites can impact microbiota interactions and host responses. Acetate can stimulate growth and metabolic activity of other mucosal bacteria and provide colonization resistance against pathogenic bacteria that cross the mucus layer to reach the intestinal cells. Propionate can signal to the host via the Gpr43 and Gpr41 receptors. This can trigger a cascade of responses in the host expression machinery and together with other signalling pathways can result in immune regulation and metabolic signalling. In addition, while Akkermansia do not produce butyrate as an end product of mucin degradation, the importance of metabolic cross-feeding on acetate by butyrate-producing bacteria in the gut has been demonstrated. Butyrate is the primary energy source for colonocytes and has been reported to regulate the physical and functional integrity of the normal colonic mucosa by altering mucin gene and tight junction expression. Additionally, butyrate has immunomodulatory effects keeping the immune cells in balance and can impact the expression of brain-derived neurotrophic factor and glia-derived neurotrophic factor leading to neuron regulation.

There is a need, therefore, for means, preferably orally or enterally administered means, more preferably dietetic means, for effectively increasing the abundance of Akkermansia in the gastro-intestinal tracts of humans, preferably non-infant humans.

### SUMMARY OF THE INVENTION

A first aspect of this invention relates to a human milk oligosaccharide (HMO) for use in increasing the abundance of Akkermansia in the gastro-intestinal tract of a human. Preferably, the HMO is for use in increasing the abundance of Akkermansia in the gastro-intestinal tract of a human to treat or prevent in the human:
an enteropathogenic infection,
metabolic disorders associated with obesity, diabetes and liver diseases,
impaired gut barrier function,
food intolerance/sensitivity such as non-coeliac wheat sensitivity,
brain gut disorders such as stress, anxiety and depressive like behaviour,
autism like behaviour, and/or
an inflammation related to a gastro-intestinal condition.

A second aspect of the invention is a synthetic composition comprising a human milk oligosaccharide for use in increasing the abundance of Akkermansia in of the gastro-intestinal tract of a human, preferably to treat or prevent in the human:
an enteropathogenic infection,
metabolic disorders associated with obesity, diabetes and liver diseases,
impaired gut barrier function,
food intolerance/sensitivity such as non-coeliac wheat sensitivity,
brain gut disorders such as stress, anxiety and depressive like behaviour,
autism-like behaviour, and/or
an inflammation related to a gastro-intestinal condition.

The synthetic composition can be a nutritional or pharmaceutical composition.

The HMO can be a neutral HMO or an acidic HMO. The neutral HMO can be one or more fucosylated HMOs or one or more non-fucosylated HMOs. Preferably, the HMO is 2′-FL, 3-FL, DFL, LNT, LNnT, 3′-SL, 6′-SL, LNFP-I or a mixture thereof. Preferably, the HMO comprises, consists of or essentially consists of 2′-FL and at least one of LNnT and LNT; at least one of 2′-FL and DFL and at least one of LNnT and LNT (e.g. 2′-FL, DFL and at least one of LNnT and LNT); 2′-FL and 6′-SL; DFL and 6′-SL; 2′-FL, DFL and 6′-SL; 2′-FL, 6′-SL and at least one of LNnT and LNT; and 2′-FL, DFL, 6′-SL and at least of LNnT and LNT.

A third aspect of this invention is a method for increasing the abundance of Akkermansia in the gastro-intestinal tract of a human, the method comprising orally or enterally administering to the human an effective amount of a human milk oligosaccharide (HMO). Preferably, the abundance of Akkermansia is increased in the mucosal layer of the gastro-intestinal track; more preferably in the colon; for example, the distal colon. Preferably, the HMO is administered to the human for a period of at least about 14 days more preferably at least about 21 days. Also, preferably, the human is administered an amount of about 1 g to about 15 g per day of the HMO, more preferably about 2 g to about 10 g per day. For example, the human may be administered about 3 g to about 7 g per day. “About” means+/−5%.

In one embodiment of the third aspect, the method comprises enterally, preferably orally, administering to a human, preferably a non-infant human:

(a) in a first step for a period of at least about 7 days (also referred to as treatment or initial treatment phase):
a first amount of a human milk oligosaccharide, or
a synthetic composition comprising a first amount of a human milk oligosaccharide,
   wherein the first amount is effective to increase the abundance of Akkermansia in the gastro-intestinal tract of the human, and

(b) in a second step for an additional period, preferably of at least about 7 days (also referred to as maintenance phase):
a second amount of a human milk oligosaccharide, or
a synthetic composition comprising a second amount of a human milk oligosaccharide,
   wherein the second amount is effective to maintain the abundance of Akkermansia in the gastro-intestinal tract of the human. Preferably, the HMO(s) is administered in the first step for a period of at least about 14 days, more preferably at least about 21 days, for example up to about 28 days. Also preferably, the additional period in the second step is at least about 21 days, for example at least about 28 days. The patient may be administered higher doses during the first step and lower doses during the second step. The dose administered during the first step is preferably about 3 g to about 10 g per day (for example about 4 g to about 7.5 g per day) and the dose administered during the second step is preferably about 2 g to about 7.5 g per day (for example about 2 g to about 5 g per day).

A fourth aspect of this invention is a method for the prophylaxis or treatment of an enteropathogenic infection in a human, the method comprising orally or enterally administering to the human, an amount of one or more human milk oligosaccharides effective to increase the abundance of Akkermansia in the gastro-intestinal tract of the human.

A fifth aspect of this invention is a method for the prophylaxis or treatment of a human having type 2 diabetes, obesity and/or liver disease, the method comprising orally or enterally administering to the human, an amount of one or more human milk oligosaccharides effective to increase abundance of Akkermansia, in the gastro-intestinal tract of the human. Preferably the amount administered is sufficient to improve intestinal permeability and/or increase insulin sensitivity and/or promote weight loss in the subject.

A sixth aspect of this invention is a method for the prophylaxis or treatment of a human having an inflammation related gastro-intestinal condition, the method comprising orally or enterally administering to the human an amount of one or more human milk oligosaccharides effective to increase abundance of Akkermansia, in the gastro-intestinal tract of the human. The gastro-intestinal condition may be intestinal bowel disease or irritable bowel syndrome. Preferably, the amount of the HMO(s) is sufficient to induce an anti-inflammatory immune response.

A seventh aspect of this invention is a method for the prophylaxis or treatment of a human having a brain gut disorder, the method comprising orally or enterally administering to the human, an amount of one or more human milk oligosaccharides effective to increase the abundance of Akkermansia in the gastro-intestinal tract of the human. The brain gut disorder may be stress, anxiety and depressive like behaviour.

An eight aspect of this invention is a method for the prophylaxis or treatment of a human having a food intolerance/sensitivity, the method comprising orally or enterally administering to the human, an amount of one or more human milk oligosaccharides effective to increase the abundance of Akkermansia in the gastro-intestinal tract of the human. In one embodiment, the food intolerance/sensitivity may be non-coeliac wheat sensitivity.

A ninth aspect of this invention is a method for the prophylaxis or treatment of a human having impaired gut barrier function, the method comprising orally or enterally administering to the human an amount of one or more human milk oligosaccharides effective to increase the abundance of Akkermansia in the gastro-intestinal tract of the human.

A tenth aspect of this invention is a method for the prophylaxis or treatment of a human having autism-like behaviour, the method comprising orally or enterally administering to the human an amount of one or more human milk oligosaccharides effective to increase the abundance of Akkermansia in the gastro-intestinal tract of the human.

In any of the fourth to tenth aspects of the invention, the HMO is preferably administered to the human for a period of at least about 14 days, more preferably at least about 21 days.

In any of the fourth to tenth aspects of the invention, the human is preferably administered an amount of 1 g to 15 g per day of the HMO, more preferably 2 g to 10 g per day. For example, the human may be administered 3 g to 7 g per day.

An eleventh aspect of this invention relates to a pack comprising at least 14 individual daily doses of an effective amount of at least one human milk oligosaccharide (HMO) for use in increasing the abundance of Akkermansia in the gastro-intestinal tract of a human, preferably to treat or prevent in the human
an enteropathogenic infection,
metabolic disorders associated with obesity, diabetes and liver diseases,
impaired gut barrier function,
food intolerance/sensitivity such as non-coeliac wheat sensitivity,
brain gut disorders such as stress, anxiety and depressive like behaviour,
autism like behaviour, and/or
an inflammation related to a gastro-intestinal condition.

Preferably, each dose contains about 1 g to about 15 g of the human milk oligosaccharide, more preferably about 2 g to about 10 g, for example, about 3 g to about 7 g. Preferably, the pack comprises at least 21 individual daily doses, more preferably at least 28 daily doses, for example, at least 35 daily doses. The pack can include instructions for use.

A twelfth aspect of the invention is a use of
one or more human milk oligosaccharides (HMOs),
a synthetic composition comprising one or more human milk oligosaccharides (HMOs), or
a pack comprising at least 14 individual daily doses of an effective amount of one or more human milk oligosaccharides
   in the dietary management of a patient suffering from one or more of the following:
an enteropathogenic infection,
metabolic disorders associated with obesity, diabetes and liver diseases,
impaired gut barrier function,
food intolerance/sensitivity such as non-coeliac wheat sensitivity,
brain gut disorders such as stress, anxiety and depressive like behaviour,
autism like behaviour,
an inflammation related to a gastro-intestinal condition.

In all aspects of the invention, the human is preferably a non-infant human.

### DESCRIPTION OF FIGURES

FIG. 1 shows the percentage of the change in Akkermansia abundance in nine treatment groups compared to the placebo group in a human clinical trial (see Example 1).

FIG. 2 shows the percentage of the change in Akkermansia in an in vitro intestine model when HMOs have been fed during a treatment period of 3 weeks.

### DETAILED DESCRIPTION OF THE INVENTION

It has now been surprisingly found that administration of human milk oligosaccharides (HMOs) to humans preferentially increases the abundance of a Akkermansia, in the microbiota of their gastro-intestinal tract. It has been previously reported in WO 2016/138911 that the administration of HMOs to a human subject increases the abundance of bifidobacteria of the B. adolescentis phylogenetic group, especially Bifidobacterium adolescentis and/or Bifidobacterium pseudocatenulatum. This increase in the bifidobacteria of the B. adolescentis phylogenetic group is temporary and lasts about 14 days. Thereafter, the abundance of Bifidobacterium longum and/or Bifidobacterium bifidum increases. It has now been surprisingly found that administration of HMO(s) to a human stimulates growth of Akkermansia in the gastro-intestinal tract of the human raising the level of Akkermansia up to 2-10 times, such as 3-5 times, compared to the level of Akkermansia before the beginning of administration of HMO(s).

Thus, it has been discovered that human milk oligosaccharides, by oral or enteral ingestion, dynamically modulate the human intestinal microbiota by preferentially promoting the growth of Akkermansia in addition to Bifidobacterium in the human intestine. As an outcome, a more beneficial intestinal microbial community and intestinal environment can be shaped and maintained, and by the increased abundance of Akkermansia, pathogenic infections can be inhibited, and intestinal and extra-intestinal diseases can be prevented or improved. The increase of Akkermansia also occurs in the mucosal layer and not only the lumen of the intestine.

Herein, the following terms have the following meanings:

“Non-infant human” or “non-infant” means a human of 3 years of age and older. A non-infant human can be a child, a teenager, an adult or an elderly person.

“Human milk oligosaccharide” or “HMO” means a complex carbohydrate found in human breast milk (Urashima et al.: Milk Oligosaccharides. Nova Science Publisher (2011); Chen Adv. Carbohydr. Chem. Biochem. 72, 113 (2015)). The HMOs have a core structure comprising a lactose unit at the reducing end that can be elongated by one or more β-N-acetyl-lactosaminyl and/or one or β-more lacto-N-biosyl units, and which core structure can be substituted by an α L-fucopyranosyl and/or an α-N-acetyl-neuraminyl (sialyl) moiety. In this regard, the non-acidic (or neutral) HMOs are devoid of a sialyl residue, and the acidic HMOs have at least one sialyl residue in their structure. The non-acidic (or neutral) HMOs can be fucosylated or non-fucosylated. Examples of such neutral non-fucosylated HMOs include lacto-N-tetraose (LNT), lacto-N-neotetraose (LNnT), lacto-N-neohexaose (LNnH), para-lacto-N-neohexaose (pLNnH), para-lacto-N-hexaose (pLNH) and lacto-N-hexaose (LNH). Examples of neutral fucosylated HMOs include 2′-fucosyllactose (2′-FL), lacto-N-fucopentaose I (LN FP-I), lacto-N-difucohexaose I (LNDFH-I), 3-fucosyllactose (3-FL), difucosyllactose (DFL), lacto-N-fucopentaose II (LNFP-II), lacto-N-fucopentaose III (LNFP-III), lacto-N-difucohexaose III (LNDFH-III), fucosyl-lacto-N-hexaose II (FLNH-II), lacto-N-fucopentaose V (LNFP-V), lacto-N-fucopentaose VI (LNFP-VI), lacto-N-difucohexaose II (LNDFH-II), fucosyl-lacto-N-hexaose I (FLNH-I), fucosyl-para-lacto-N-hexaose I (FpLNH-I), fucosyl-para-lacto-N-neohexaose II (F-pLNnH II) and fucosyl-lacto-N-neohexaose (FLNnH). Examples of acidic HMOs include 3′-sialyllactose (3′-SL), 6′-sialyllactose (6′-SL), 3-fucosyl-3′-sialyllactose (FSL), LST a, fucosyl-LST a (FLST a), LST b, fucosyl-LST b (FLST b), LST c, fucosyl-LST c (FLST c), sialyl-LNH (SLNH), sialyl-lacto-N-hexaose (SLNH), sialyl-lacto-N-neohexaose I (SLNH-1), sialyl-lacto-N-neohexaose 11 (SLNH-II) and disialyl-lacto-N-tetraose (DSLNT).

“Synthetic composition” means a composition which is artificially prepared and preferably means a composition containing at least one compound that is produced ex vivo chemically and/or biologically, e.g. by means of chemical reaction, enzymatic reaction or recombinantly. In some embodiments, a synthetic composition of the invention may be, but preferably is not, identical with a naturally occurring composition. The synthetic composition typically comprises one or more compounds, advantageously HMOs, that are capable of preferentially increasing the abundance of Akkermansia in the gastro-intestinal tract of the human. In some embodiments, the synthetic composition may comprise one or more compounds or components other than HMOs that may have a beneficial effect on the microbiota of a human subject microbiota in vivo, e.g. non-digestible oligosaccharides or prebiotics. Also, in some embodiments, the synthetic compositions may comprise one or more nutritionally or pharmaceutically active components which do not affect adversely the efficacy of the above-mentioned compounds. Some non-limiting embodiments of a synthetic composition of the invention are also described below.

“Microbiota”, “microflora” and “microbiome” mean a community of living microorganisms that typically inhabits a bodily organ or part, particularly the gastro-intestinal organs of non-infant humans. The most dominant members of the gastrointestinal microbiota include microorganisms of the phyla of Firmicutes, Bacteroidetes, Actinobacteria, Proteobacteria, Synergistetes, Verrucomicrobia, Fusobacteria, and Euryarchaeota; at genus level Bacteroides, Faecalibacterium, Bifidobacterium, Roseburia, Alistipes, Collinsella, Blautia, Coprococcus, Ruminococcus, Eubacterium and Dorea; at species level Bacteroides uniformis, Alistipes putredinis, Parabacteroides merdae, Ruminococcus bromii, Dorea longicatena, Bacteroides caccae, Bacteroides thetaiotaomicron, Eubacterium hallii, Ruminococcus torques, Faecalibacterium prausnitzii, Ruminococcus lactaris, Collinsella aerofaciens, Dorea formicigenerans, Bacteroides vulgatus and Roseburia intestinalis. The gastrointestinal microbiota includes the mucosa-associated microbiota, which is located in or attached to the mucus layer covering the epithelium of the gastrointestinal tract, and luminal-associated microbiota, which is found in the lumen of the gastrointestinal tract.

“Enteral administration” means any conventional form for delivery of a composition to a human that causes the deposition of the composition in the gastrointestinal tract (including the stomach). Methods of enteral administration include feeding through a naso-gastric tube or jejunum tube, oral, sublingual and rectal.

“Oral administration” means any conventional form for the delivery of a composition to a human through the mouth. Accordingly, oral administration is a form of enteral administration.

“Effective amount” means an amount of a composition that provides an HMO in a sufficient amount to render a desired treatment outcome in a human. An effective amount can be administered in one or more doses to achieve the desired treatment outcome.

“Relative abundance” of a bacterial species means the abundance of that species relative to other bacteria in the microbiota of the gastro-intestinal tract of humans.

“Relative growth” of a bacterial species means the growth of that species relative to other bacteria in the microbiota in the gastro-intestinal tract of humans.

“Bifidobacterium of the B. adolescentis phylogenetic group” means a bacterium selected from a group consisting of Bifidobacterium adolescentis, Bifidobacterium angulatum, Bifidobacterium catenulatum, Bifidobacterium pseudocatenulatum, Bifidobacterium kashiwanohense, Bifidobacterium dentum and Bifidobacterium stercoris (Duranti et al. Appl. Environ. Microbiol. 79, 336 (2013), Bottacini et al. Microbial Cell Fact. 13:S4 (2014)). Preferably, a Bifidobacterium of the B. adolescentis phylogenetic group is Bifidobacterium adolescentis and/or Bifidobacterium pseudocatenulatum.

“Treat” means to address a medical condition or disease with the objective of improving or stabilising an outcome in the person being treated or addressing an underlying nutritional need. Treat, therefore, includes the dietary or nutritional management of the medical condition or disease by addressing nutritional needs of the person being treated. “Treating” and “treatment” have grammatically corresponding meanings.

“Modulating of microbiota” means exerting a modifying or controlling influence on microbiota, for example an influence leading to an increase in the indigenous intestinal abundance of Bifidobacterium, Barnesiella, Faecalibacterium and/or other butyrate producing bacteria. In another example, the influence may lead to a reduction of the intestinal abundance of Ruminococcus gnavus and/or Proteobacteria. “Proteobacteria” are a phylum of Gram-negative bacteria and include a wide variety of pathogenic bacteria, such as Escherichia, Salmonella, Vibrio, Helicobacter, Yersinia and many other notable genera.

“Therapy” means treatment given or action taken to reduce or eliminate symptoms of a disease or pathological condition.

“Preventive treatment” or “prevention” means treatment given or action taken to diminish the risk of onset or recurrence of a disease.

“Secondary prevention” means prevention of onset of the condition in a high-risk patient, or prevention of reoccurrence of symptoms in a patient who has already has the condition. A “high-risk” patient is an individual who is predisposed to developing the condition; for example a person with a family history of the condition.

“Dietary management” means exclusive or partial feeding of patients who, because of a disease, disorder or medical condition are suffering from:
either have a limited, impaired or disturbed capacity to take, digest, absorb, metabolise or excrete ordinary food or certain nutrients contained therein, or metabolites, or
have other medically-determined nutrient requirements
   (see: Commission Notice on the classification of Food for Special Medical Purposes of the European Commission, Official Journal of the European Union C 401, 25.11.2017, p. 10-11).

In accordance with this invention, it has been discovered that an HMO can stimulate the growth of Akkermansia in the gastro-intestinal tract of humans, particularly when administered to the humans over several days, for example for at least about 14 days. For this reason, an HMO can be used for increasing the abundance of Akkermansia in the gastro-intestinal tract of humans. Accordingly, an HMO can be used for treating or preventing viral and/or bacterial infections (especially enteropathogenic infections), intestinal inflammatory diseases (especially IBD), IBS, gut-brain disorders, and extra-intestinal diseases: metabolic disorders (such as obesity and type 2 diabetes and the associated co-morbidities, such as glucose intolerance, abnormal lipid metabolism, atherosclerosis, hypertension, cardiac pathology, stroke, dysfunction of the immune system, high cholesterol, elevated triglycerides); liver diseases (such as non-alcoholic fatty liver disease, hyperglycaemia, hepatic steatosis, dyslipidaemia); asthma; sleep apnoea; osteoarthritis; neuro-degeneration; gallbladder disease; syndrome X; inflammatory and immune disorders; atherogenic dyslipidaemia; cancer, in particular gut, intestine and colon cancers; autism and food intolerance/sensitivity, in humans.

Accordingly, the first aspect of the invention relates to an HMO for use for the increase in the abundance of Akkermansia in the gastro-intestinal tract of humans, and thereby for treating and/or preventing viral and/or bacterial infections (especially enteropathogenic infections), intestinal inflammatory diseases (especially IBD), IBS and gut-brain disorders and extra-intestinal diseases (especially obesity and type 2 diabetes, liver disease, autism and food intolerance/sensitivity) in humans.

The second aspect of this invention is a synthetic composition comprising an HMO for use in increasing the abundance of Akkermansia in the gastro-intestinal tract of a human, and thereby treating and/or preventing viral and/or bacterial infections (especially enteropathogenic infections), intestinal inflammatory diseases (especially IBD), IBS and gut-brain disorders and extra-intestinal diseases (especially obesity and type 2 diabetes, liver disease, autism and food intolerance/sensitivity).

A third aspect of this invention is a method for increasing the abundance of Akkermansia in the gastro-intestinal tract of a human, the method comprising orally or enterally administering to the human an effective amount of a human milk oligosaccharide (HMO).

A fourth aspect of this invention is a method for the prophylaxis or treatment of an enteropathogenic infection in a human, the method comprising orally or enterally administering to the human, an amount of one or more human milk oligosaccharides effective to increase the abundance of Akkermansia in the gastro-intestinal tract of the human.

A fifth aspect of this invention is a method for the prophylaxis or treatment of a human having type 2 diabetes, obesity and/or liver disease, the method comprising orally or enterally administering to the human, an amount of one or more human milk oligosaccharides effective to increase abundance of Akkermansia, in the gastro-intestinal tract of the human.

A sixth aspect of this invention is a method for the prophylaxis or treatment of a human having an inflammation related gastro-intestinal condition, the method comprising orally or enterally administering to the human an amount of one or more human milk oligosaccharides effective to increase abundance of Akkermansia, in the gastro-intestinal tract of the human. The gastro-intestinal condition may be intestinal bowel disease or irritable bowel syndrome.

A seventh aspect of this invention is a method for the prophylaxis or treatment of a human having a gut-brain disorder, the method comprising orally or enterally administering to the human, an amount of one or more human milk oligosaccharides effective to increase the abundance of Akkermansia in the gastro-intestinal tract of the human. The brain gut disorder may be stress, anxiety and depressive like behaviour.

An eight aspect of this invention is a method for the prophylaxis or treatment of a human having a food intolerance/sensitivity, the method comprising orally or enterally administering to the human, an amount of one or more human milk oligosaccharides effective to increase the abundance of Akkermansia in the gastro-intestinal tract of the human. In one embodiment, the food intolerance/sensitivity may be non-coeliac wheat sensitivity.

A ninth aspect of this invention is a method for the prophylaxis or treatment of a human having impaired gut barrier function, the method comprising orally or enterally administering to the human an amount of one or more human milk oligosaccharides effective to increase the abundance of Akkermansia in the gastro-intestinal tract of the human.

A tenth aspect of this invention is a method for the prophylaxis or treatment of a human having autism-like behaviour, the method comprising orally or enterally administering to the human an amount of one or more human milk oligosaccharides effective to increase the abundance of Akkermansia in the gastro-intestinal tract of the human.

An eleventh aspect of this invention relates to a pack comprising at least 14 individual daily doses of an effective amount of at least one human milk oligosaccharide (HMO) for use in increasing the abundance of Akkermansia in the gastro-intestinal tract of a human, preferably to treat or prevent in the human
an enteropathogenic infection,
metabolic disorders associated with obesity, diabetes and liver diseases,
impaired gut barrier function,
food intolerance/sensitivity such as non-coeliac wheat sensitivity,
brain gut disorders such as stress, anxiety and depressive like behaviour,
autism like behaviour, and/or
an inflammation related to a gastro-intestinal condition.

A twelfth aspect of the invention is a use of
one or more human milk oligosaccharides (HMOs),
a synthetic composition comprising one or more human milk oligosaccharides (HMOs), or
a pack comprising at least 14 individual daily doses of an effective amount of one or more human milk oligosaccharides
   in the dietary management of a patient suffering from one or more of the following:
an enteropathogenic infection,
metabolic disorders associated with obesity, diabetes and liver diseases,
impaired gut barrier function,
food intolerance/sensitivity such as non-coeliac wheat sensitivity,
brain gut disorders such as stress, anxiety and depressive like behaviour,
autism like behaviour,
an inflammation related to a gastro-intestinal condition.

Concerning each aspect, the HMOs suitable for use in increasing the abundance of Akkermansia in the gastro-intestinal tract of humans, can be isolated or enriched by well-known processes from milk(s) secreted by mammals including, but not limited to human, bovine, ovine, porcine, or caprine species. The HMOs can also be produced by well-known processes using microbial fermentation, enzymatic processes, chemical synthesis, or combinations of these technologies. As examples, using chemistry LNnT can be made as described in WO 2011/100980 and WO 2013/044928, LNT can be synthesized as described in WO 2012/155916 and WO 2013/044928, a mixture of LNT and LNnT can be made as described in WO 2013/091660, 2′-FL can be made as described in WO 2010/115934 and WO 2010/115935, 3-FL can be made as described in WO 2013/139344, 6′-SL and salts thereof can be made as described in WO 2010/100979, sialylated oligosaccharides can be made as described in WO 2012/113404 and mixtures of human milk oligosaccharides can be made as described in WO 2012/113405. As examples of enzymatic production, sialylated oligosaccharides can be made as described in WO 2012/007588, fucosylated oligosaccharides can be made as described in WO 2012/127410, and advantageously diversified blends of human milk oligosaccharides can be made as described in WO 2012/156897 and WO 2012/156898. Biotechnological methods which describe how to make core human milk oligosaccharides optionally substituted by fucose or sialic acid using genetically modified E. coli can be found in WO 01/04341 and WO 2007/101862.

The HMO in any of the above aspects may be a single HMO or a mixture of any HMOs suitable for the purpose of the invention. The HMO can be a neutral HMO or an acidic HMO. The neutral HMO is, in one embodiment, one or more fucosylated HMOs; in another embodiment, the neutral HMO is one or more non-fucosylated HMOs. Particularly, the fucosylated neutral HMO is selected from the list consisting of 2′-FL, 3-FL, DFL, LNFP-I, LNFP-II, LNFP-III, LNFP-V, LNFP-VI, LNDFH-I, LNDFH-II, LNDFH-III, FLNH-I, FLNH-II, FLNnH, FpLNH-I and F-pLNnH II, preferably, 2′-FL, and the non-fucosylated neutral HMO is selected from the list consisting of LNT, LNnT, LNH, LNnH, pLNH and pLNnH, e.g. LNnT. The one or more fucosylated HMOs can be e.g. a mixture containing, consisting or consisting essentially of 2′-FL and DFL.

In one embodiment, the mixture comprises, consists of or essentially consists of, neutral HMOs, preferably at least a first neutral HMO and at least a second neutral HMO, wherein the first neutral HMO is a fucosylated neutral HMO and the second neutral HMO is a non-fucosylated neutral HMO. The fucosylated neutral HMO(s) and the non-fucosylated neutral HMO(s) may be present in a mass ratio of about 4:1 to 1:1. Particularly, the mixture of HMOs comprises, consists of or essentially consists of a fucosylated HMO selected from the list consisting of 2′-FL, 3-FL, DFL, LNFP-I, LNFP-II, LNFP-III, LNFP-V, LNDFH-I, LNDFH-II, LNDFH-III, FLNH-I, FLNH-II, FLNnH, FpLNH-I and F-pLNnH II, and a non-fucosylated neutral HMO selected from the list consisting of LNT, LNnT, LNH, LNnH, pLNH and pLNnH. More preferably, the mixture of neutral HMOs contains, consists of or essentially consists of, a fucosylated HMO selected from the list consisting of 2′-FL, 3-FL and DFL, and a non-fucosylated neutral HMO selected from the list consisting of LNT and LNnT; advantageously the mixture comprises, consists of or essentially consists of, 2′-FL and at least one of LNnT and LNT; or at least one of 2′-FL and DFL and at least one of LNnT and LNT; or 2′-FL, DFL and at least one of LNnT and LNT.

In other embodiment, the mixture comprises, consists of or essentially consists of, at least a first (acidic) HMO and at least a second (neutral) HMO, wherein the first (acidic) HMO is selected from the list consisting of 3′-SL, 6′-SL and FSL and the second (neutral) HMO is selected from the list consisting of 2′-FL, 3-FL, DFL, LNT and LNnT; advantageously the mixture comprises, consists of or essentially consists of, 2′-FL and 6′-SL; or 6′-SL and at least one of 2′-FL and DFL; or 2′-FL, 6′-SL and at least one of LNnT and LNT; or 2′-FL, DFL, 6′-SL and at least one of LNnT and/or LNT.

The synthetic composition can be a pharmaceutical composition. The pharmaceutical composition can contain a pharmaceutically acceptable carrier, e.g. phosphate buffered saline solution, mixtures of ethanol in water, water and emulsions such as an oil/water or water/oil emulsion, as well as various wetting agents or excipients. The pharmaceutical composition can also contain other materials that do not produce an adverse, allergic or otherwise unwanted reaction when administered to humans. The carriers and other materials can include solvents, dispersants, coatings, absorption promoting agents, controlled release agents, and one or more inert excipients, such as starches, polyols, granulating agents, microcrystalline cellulose, diluents, lubricants, binders, and disintegrating agents. If desired, tablet dosages of the anti-infective compositions can be coated by standard aqueous or non-aqueous techniques.

The pharmaceutical compositions can be administered orally, e.g. as a tablet, capsule, or pellet containing a predetermined amount, or as a powder or granules containing a predetermined concentration or a gel, paste, solution, suspension, emulsion, syrup, bolus, electuary, or slurry, in an aqueous or non-aqueous liquid, containing a predetermined concentration. Orally administered compositions can include binders, lubricants, inert diluents, flavouring agents, and humectants. Orally administered compositions such as tablets can optionally be coated and can be formulated to provide sustained, delayed or controlled release of the mixture therein.

The pharmaceutical compositions can also be administered by rectal suppository, aerosol tube, naso-gastric tube or direct infusion into the GI tract or stomach.

The pharmaceutical compositions can also include therapeutic agents such as antiviral agents, antibiotics, probiotics, analgesics, and anti-inflammatory agents. The proper dosage of these compositions for a human can be determined in a conventional manner, based upon factors such immune status, body weight and age. In some cases, the dosage will be at a concentration similar to that found for the HMO in human breast milk. The required amount would generally be in the range from about 200 mg to about 20 g per day, in certain embodiments from about 300 mg to about 15 g per day, from about 400 mg to about 10 g per day, in certain embodiments from about 500 mg to about 10 g per day, in certain embodiments from about 1 g to about 10 g per day. Appropriate dose regimes can be determined by conventional methods.

The synthetic composition can also be a nutritional composition. It can contain sources of protein, lipids and/or digestible carbohydrates and can be in powdered or liquid forms. The composition can be designed to be the sole source of nutrition or a nutritional supplement.

Suitable protein sources include milk proteins, soy protein, rice protein, pea protein and oat protein, or mixtures thereof. Milk proteins can be in the form of milk protein concentrates, milk protein isolates, whey protein or casein, or mixtures of both. The protein can be whole protein or hydrolysed protein, either partially hydrolysed or extensively hydrolysed. Hydrolysed protein offers the advantage of easier digestion which can be important for humans with inflamed GI tracts. The protein can also be provided in the form of free amino acids. The protein can comprise about 5% to about 30% of the energy of the nutritional composition, normally about 10% to 20%.

The protein source can be a source of glutamine, threonine, cysteine, serine, proline, or a combination of these amino acids. The glutamine source can be a glutamine dipeptide and/or a glutamine enriched protein. Glutamine can be included due to the use of glutamine by enterocytes as an energy source. Threonine, serine and proline are important amino acids for the production of mucin. Mucin coats the GI tract and can improve mucosal healing. Cysteine is a major precursor of glutathione, which is key for the antioxidant defences of the body.

Suitable digestible carbohydrates include maltodextrin, hydrolysed or modified starch or corn starch, glucose polymers, corn syrup, corn syrup solids, high fructose corn syrup, rice-derived carbohydrates, pea-derived carbohydrates, potato-derived carbohydrates, tapioca, sucrose, glucose, fructose, sucrose, lactose, honey, sugar alcohols (e.g., maltitol, erythritol, sorbitol), or mixtures thereof. Generally digestible carbohydrates provide about 35% to about 55% of the energy of the nutritional composition. Preferably the nutritional composition is free from lactose. A particularly suitable digestible carbohydrate is a low dextrose equivalent (DE) maltodextrin.

Suitable lipids include medium chain triglycerides (MCT) and long chain triglycerides (LCT). Preferably the lipid is a mixture of MCTs and LCTs. For example, MCTs can comprise about 30% to about 70% by weight of the lipids, more specifically about 50% to about 60% by weight. MCTs offer the advantage of easier digestion which can be important for humans with inflamed GI tracts. Generally, the lipids provide about 35% to about 50% of the energy of the nutritional composition. The lipids can contain essential fatty acids (omega-3 and omega-6 fatty acids). Preferably these polyunsaturated fatty acids provide less than about 30% of total energy of the lipid source. Decreasing the levels of these polyunsaturated fatty acids is believed to decrease sensitivity to peroxidation; which can be beneficial for humans having inflammatory conditions.

Suitable sources of long chain triglycerides are rapeseed oil, sunflower seed oil, palm oil, soy oil, milk fat, corn oil, high oleic oils, and soy lecithin. Fractionated coconut oils are a suitable source of medium chain triglycerides. The lipid profile of the nutritional composition is preferably designed to have a polyunsaturated fatty acid omega-6 (n-6) to omega-3 (n-3) ratio of about 4:1 to about 10:1. For example, the n-6 to n-3 fatty acid ratio can be about 6:1 to about 9:1.

The nutritional composition preferably also includes vitamins and minerals. If the nutritional composition is intended to be a sole source of nutrition, it preferably includes a complete vitamin and mineral profile. Examples of vitamins include vitamins A, B-complex (such as B1, B2, B6 and B12), C, D, E and K, niacin and acid vitamins such as pantothenic acid, folic acid and biotin. Examples of minerals include calcium, iron, zinc, magnesium, iodine, copper, phosphorus, manganese, potassium, chromium, molybdenum, selenium, nickel, tin, silicon, vanadium and boron.

The nutritional composition can also include a carotenoid such as lutein, lycopene, zeaxanthin, and beta-carotene. The total amount of carotenoid included can vary from about 0.001 μg/ml to about 10 μg/ml. Lutein can be included in an amount of from about 0.001 μg/ml to about 10 μg/ml, preferably from about 0.044 μg/ml to about 5 g/ml of lutein. Lycopene can be included in an amount from about 0.001 μg/ml to about 10 μg/ml, preferably about 0.0185 mg/ml to about 5 g/ml of lycopene. Beta-carotene can comprise from about 0.001 μg/ml to about 10 mg/ml, for example about 0.034 μg/ml to about 5 μg/ml of beta-carotene.

The nutritional composition preferably also contains reduced concentrations of sodium; for example, from about 300 mg/l to about 400 mg/l. The remaining electrolytes can be present in concentrations set to meet needs without providing an undue renal solute burden on kidney function. For example, potassium is preferably present in a range of about 1180 to about 1300 mg/l; and chloride is preferably present in a range of about 680 to about 800 mg/l.

The nutritional composition can also contain various other conventional ingredients such as preservatives, emulsifying agents, thickening agents, buffers, fibres and prebiotics (e.g. fructooligosaccharides, galactooligosaccharides), probiotics (e.g. B. animalis subsp. lactis BB-12, B. lactis HNO19, B. lactis Bi07, B. infantis ATCC 15697, L. rhamnosus GG, L. rhamnosus HNOOI, L. acidophilus LA-5, L. acidophilus NCFM, L. fermentum CECT5716, B. longum BB536, B. longum AH1205, B. longum AH1206, B. breve M-16V, L. reuteri ATCC 55730, L. reuteri ATCC PTA-6485, L. reuteri DSM 17938), antioxidant/anti-inflammatory compounds including tocopherols, carotenoids, ascorbate/vitamin C, ascorbyl palmitate, polyphenols, glutathione, and superoxide dismutase (melon), other bioactive factors (e.g. growth hormones, cytokines, TFG-13), colorants, flavours, and stabilisers, lubricants, and so forth.

The nutritional composition can be in the form of a soluble powder, a liquid concentrate, or a ready-to-use formulation. The composition can be fed to a human via a nasogastric tube or orally. Various flavours, fibres and other additives can also be present.

The nutritional compositions can be prepared by any commonly used manufacturing techniques for preparing nutritional compositions in solid or liquid form. For example, the composition can be prepared by combining various feed solutions. A protein-in-fat feed solution can be prepared by heating and mixing the lipid source and then adding an emulsifier (e.g. lecithin), fat soluble vitamins, and at least a portion of the protein source while heating and stirring. A carbohydrate feed solution is then prepared by adding minerals, trace and ultra trace minerals, thickening or suspending agents to water while heating and stirring. The resulting solution is held for 10 minutes with continued heat and agitation before adding carbohydrates (e.g. the HMOs and digestible carbohydrate sources). The resulting feed solutions are then blended together while heating and agitating and the pH adjusted to 6.6-7.0, after which the composition is subjected to high-temperature short-time processing during which the composition is heat treated, emulsified and homogenized, and then allowed to cool. Water soluble vitamins and ascorbic acid are added, the pH is adjusted to the desired range if necessary, flavours are added, and water is added to achieve the desired total solid level.

For a liquid product, the resulting solution can then be aseptically packed to form an aseptically packaged nutritional composition. In this form, the nutritional composition can be in ready-to-feed or concentrated liquid form. Alternatively, the composition can be spray-dried and processed and packaged as a reconstitutable powder.

When the nutritional product is a ready-to-feed nutritional liquid, the total concentration of HMOs in the liquid, by weight of the liquid, is from about 0.0001% to about 2.0%, including from about 0.001% to about 1.5%, including from about 0.01% to about 1.0%. When the nutritional product is a concentrated nutritional liquid, the total concentration of HMOs in the liquid, by weight of the liquid, is from about 0.0002% to about 4.0%, including from about 0.002% to about 3.0%, including from about 0.02% to about 2.0%.

The nutritional composition can also be in a unit dosage form such as a capsule, tablet or sachet. For example, the synthetic composition can be in a tablet form comprising the HMOs, and one or more additional components to aid formulation and administration, such as diluents, excipients, antioxidants, lubricants, colorants, binders, disintegrants, and the like.

Suitable diluents, excipients, lubricants, colorants, binders, and disintegrants include polyethylene, polyvinyl chloride, ethyl cellulose, acrylate polymers and their copolymers, hydroxyethyl-cellulose, hydroxypropylmethyl-cellulose (HPMC), sodium carboxymethylcellulose, polyhydroxyethyl methylacrylate (PHEMA), polyvinyl alcohol (PVA), polyvinyl pyrrolidone (PVP), polyethylene oxide (PEO), or polyacrylamide (PA), carrageenan, sodium alginate, polycarbophil, polyacrylic acid, tragacanth, methyl cellulose, pectin, natural gums, xanthan gum, guar gum, karaya gum, hypromellose, magnesium stearate, microcrystalline cellulose, and colloidal silicon dioxide. Suitable antioxidants are vitamin A, carotenoids, vitamin C, vitamin E, selenium, flavonoids, polyphenols, lycopene, lutein, lignan, coenzyme Q10 (“CoQIO”) and glutathione.

The unit dosage forms, especially those in sachet form, can also include various nutrients including macronutrients.

A first target group of this invention includes healthy humans. Their ingestion of one or more HMOs will stimulate the growth of Akkermansia in the gastro-intestinal tract of the healthy humans and increase the abundance of Akkermansia in the gastro-intestinal tract to up to 100-1000% (i.e. 2-11 fold).

A second target group of this invention includes humans with an enteropathogenic infection. Their ingestion of one or more HMOs will stimulate the growth of Akkermansia in the gastro-intestinal tract of the humans and increase the abundance of Akkermansia in the gastro-intestinal tract to up to 100-1000% (i.e. 2-11 fold), and, as a result, induce a favourable immune response against the enteropathogenic microorganism, inhibiting or treating infection.

A third target group for this invention includes obese humans, and/or lean or obese humans diagnosed with type 2 diabetes. Their ingestion of one or more HMOs will stimulate the growth of Akkermansia in the gastro-intestinal tract of the humans and increase abundance of Akkermansia in the gastro-intestinal tract to up to 100-1000% (i.e. 2-11 fold), and as a result, improves intestinal permeability and/or increases insulin sensitivity, hence reducing the pathological conditions of type 2 diabetes and/or obesity.

A fourth target group for this invention includes humans diagnosed with intestinal inflammation and associated diseases such as IBD and IBS. Their ingestion of one or more HMOs will stimulate the growth of Akkermansia in the gastro-intestinal tract of the humans and increase abundance of Akkermansia in the gastro-intestinal tract to up to 100-1000% (i.e. 2-11 fold), and as a result, contributes to immunomodulation by inducing an anti-inflammatory immune response, hence improving symptoms.

The gastro-intestinal condition is preferably intestinal bowel disease or irritable bowel syndrome.

A fifth target group for this invention includes humans diagnosed with food intolerance/sensitivity. Their ingestion of one or more HMOs will stimulate the growth of Akkermansia in the gastro-intestinal tract of the humans and increase abundance of Akkermansia in the gastro-intestinal tract to up to 100-1000% (i.e. 2-11 fold), and as a result, contributes to immunomodulation and improvement in gut barrier properties, hence improving symptoms.

A sixth target group for this invention includes humans having a gut-brain disorder, for example stress, anxiety or depressive like behaviour, or autism. Their ingestion of one or more HMOs will stimulate the growth of Akkermansia in the gastro-intestinal tract of the humans and increase abundance of Akkermansia in the gastro-intestinal tract to up to 100-1000% (i.e. 2-11 fold), hence improving symptoms.

The HMO can be administered to the human:
(a) in a first step, for a period of up to about 14 days:
a first amount of a human milk oligosaccharide, or a synthetic composition comprising a first amount of a human milk oligosaccharide, to increase the abundance of Akkermansia in the gastro-intestinal tract of the human to the level up to 100% or more, such as 200-500% higher, compared to the abundance of Akkermansia before the initiation of administration, and
(b) in a second step, for an additional period:
a second amount of a human milk oligosaccharide, or a synthetic composition comprising a second amount of a human milk oligosaccharide, to maintain the level of Akkermansia in the gastro-intestinal tract of the human achieved after the first step.

For stimulating the growth of Akkermansia in the gastro-intestinal tract of a human, the amount of HMO(s) required to be administered will vary depending upon factors such as the risk and severity of the obesity, type 2 diabetes, the inflammatory gastrointestinal condition, food intolerance/sensitivity, the gut brain disorder or the enteropathogenic infection, age, the form of the composition, and other medications being administered. However, the required amount can be readily set by a medical practitioner and would generally be in the range from about 10 mg to about 20 g per day, in certain embodiments from about 10 mg to about 15 g per day, from about 100 mg to about 10 g per day, in certain embodiments from about 500 mg to about 10 g per day, in certain embodiments from about 1 g to about 7.5 g per day. An appropriate dose can be determined based on several factors, including, for example, body weight and/or condition, the severity of type 2 diabetes, the inflammatory gastrointestinal condition or the enteropathogenic infection, being treated or prevented, other ailments and/or diseases, the incidence and/or severity of side effects and the manner of administration. Appropriate dose ranges may be determined by methods known to those skilled in the art. During an initial treatment phase (first step), the dosing can be higher (for example 200 mg to 20 g per day, preferably 500 mg to 15 g per day, more preferably 1 g to 10 g per day, in certain embodiments 2.5 g to 7.5 g per day). During a maintenance phase (second step), the dosing can be reduced (for example, 10 mg to 10 g per day, preferably 100 mg to 7.5 g per day, more preferably 500 mg to 5 g per day, in certain embodiments 1 g to 2.5 g per day).

Whilst the invention has been described with reference to a preferred embodiment, it will be appreciated that various modifications are possible within the scope of the invention.

### EXAMPLES

The working example described herein are for illustration purposes only and should not be considered as limiting.

### Example 1

A total of 100 male and female healthy adults are recruited to participate in the study. After a screening visit and run-in period of 1-2 weeks, the participants are selected and randomized into ten groups, each of 10 subjects. One group is administered a placebo product containing 2 grams of glucose. The remaining 9 groups are administered treatment product containing a) 20 g of 2′-FL, b) 10 g of 2′-FL, c) 5 g of 2′-FL, d) 20 g of LNnT, e) 10 g of LNnT, f) 5 g of LNnT, g) 20 g of a 2:1 mixture of 2′-FL and LNnT, h) 10 g of a 2:1 mixture of 2′-FL and LNnT (by weight), and i) 5 g of a 2:1 mixture of 2′-FL and LNnT (by weight) for 4 weeks. The placebo and treatment products are in powder form in a unit dosage container.

The healthy adults are eligible to participate if they are at an age between 18-60 years. All recruited participants are able and willing to understand and comply with the study procedures. Participants are excluded if: they had participated in a clinical study one month prior to screening visit; they had abnormal results in the screening tests which were clinically relevant for study participation; they are suffering for a severe disease such as malignancy, diabetes, severe coronary disease, kidney disease, neurological disease, or severe psychiatric disease or any condition which could confound the results of the study; used highly dosed probiotic supplements (yoghurt allowed) for 3 months prior to the study; they consumed antibiotic drugs 6 months prior to the study; they consumed on a regular basis any medication that might have interfered with symptom evaluation 2 weeks prior to the study; and are pregnant or lactating.

At the screening visit, medical history and concomitant medication is registered and a blood sample for safety analyses is collected. A faecal sample kit is distributed. Participants are instructed to keep their samples in the freezer until the next visit.

At the second visit, eligibility criteria are checked, and eligible subjects are randomised to the ten arms in the trial (treatment groups and placebo group). The faecal samples are collected and equipment for new samples are distributed. Participants are familiarised with an interactive internet enabled system which recorded data daily and are provided with either treatment or control products. Subjects are reminded not to change their usual diet during the study. Blood samples are collected for biomarker studies. The faecal samples are stored at −80° C. until analysis.

The study runs for 2 weeks with the participants consuming either a placebo or a treatment product daily. Participants are instructed to consume the products in the morning with breakfast. Compliance is monitored through the interactive internet enabled system.

The participants also use the system to record:
Bristol Stool Form Scale (BSFS) information.
Symptom information such as abdominal pain, abdominal discomfort, abdominal cramping, abdominal bloating, and abdominal fullness.
Additional, Gastrointestinal Symptom Rating Scale (GSRS) information.

This questionnaire includes 15 items covering five dimensions (abdominal pain, indigestion, reflux, diarrhoea, constipation) and uses a seven-graded Likert scale.

After 2 weeks, each participant has a visit with the medical team. Faecal samples and blood samples are collected. The faecal samples are stored at −80° C. until analysis. Equipment for new samples are distributed. Subjects are reminded not to change their usual diet during the study.

Blood samples are analysed simultaneously in a multiplexing format on an electrochemiluminescence platform. The following analytes are included in the panel: BUN, LDL cholesterol, HDL cholesterol, iron, triglycerides, ApoAl, ApoB, insulin, FFAs, glucagon, IL-10, IL-6 and TNF-α.

To assess the microbiota profile, DNA is extracted from the faecal samples using a 96-well PowerSoil DNA Isolation Kit (MO-BIO). A minimum of one sample-well per plate is kept empty to serve as a negative control during PCR. PCR is done with the forward primer S-D-Bact-0341-b-S-17 and reverse primer S-D-Bact-0785-a-A-21 with Illumina adapters attached (Klindworth et al. Nucleic Acids Res. 41, el (2013)). These are universal bacterial 16S rDNA primers, which targeted the V3-V4 region. The following PCR program is used: 98° C. for 30 sec, 25× (98° C. for 10 s, 55° C. for 20 s, 72° C. for 20 s), 72° C. for 5 min. Amplification is verified by running the products on a 1% agarose gel. Barcodes are added in a nested PCR using the Nextera Index Kit V2 (Illumina) with the following PCR program: 98° C. for 30 sec, 8× (98° C. for 10 s, 55° C. for 20 s, 72° C. for 20 s), 72° C. for 5 min. Attachment of primers is verified by running the products on a 1% agarose gel. Products from the nested PCR are normalized using the SequalPrep Normalization Plate Kit and pooled. Pooled libraries are concentrated by evaporation and the DNA concentration of pooled libraries is measured on a Qubit fluorometer using the Qubit High Sensitivity Assay Kit (Thermo Fisher Scientific). Sequencing is done on a MiSeq desktop sequencer using the MiSeq Reagent Kit V3 (Illumina) for 2×300 bp paired-end sequencing. The 64-bit version of USEARCH is used for bioinformatical analysis of the sequence data.

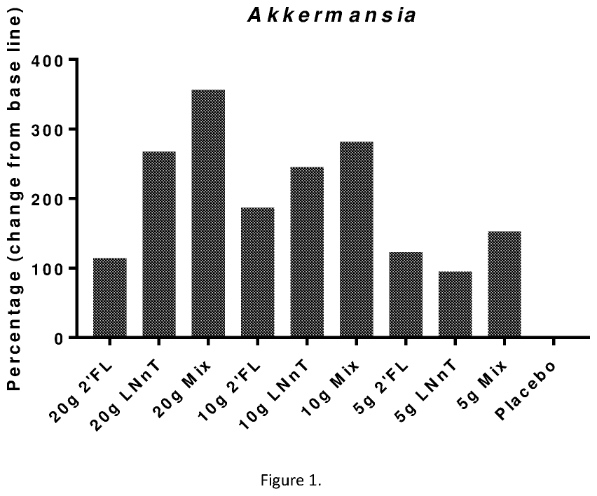

As illustrated in FIG. 1, the results from the profiling of the microbial community show that the abundance of Akkermansia increased when consuming HMO while it remained unchanged in the placebo group (values are calculated as change in percent compared to the t=0 value). This means that the oral ingestion of HMOs clearly increases the abundance of Akkermansia in the intestinal microbiota of healthy adults.

### Example 2

The impact of the HMOs on microbiota is investigated in the M-TripleSHIME™ in vitro gastrointestinal model (Prodigest). The typical reactor setup of the M-TripleSHIME™ consists of a succession of four reactors simulating the different parts of the human gastrointestinal tract. The first two reactors are of the fill-and-draw principle to simulate different steps in food uptake and digestion, with peristaltic pumps adding a defined amount of SHIME feed (140 ml 3×/day) and pancreatic and bile liquid (60 ml 3×/day), respectively, to the stomach and small intestine compartment and emptying the respective reactors after specified intervals. The last two compartments are continuously stirred reactors with constant volume and pH control. The retention time and pH of the different vessels are chosen to resemble in vivo conditions in the different parts of the colon. The proximal colon is set to pH 5.4-5.6 and retention time=12 h, and the distal colon is set to pH 6.0-6.5 and retention time=20 h. 2′-FL, LNnT or a mixture of 2′-FL and LNnT in 4:1 mass ratio is added to the SHIME feed in a concentration that equals 10 grams per day.

Upon inoculation with faecal microbiota, these reactors simulate the proximal, transverse and distal colon. After a two-week adaptation of the microbial communities in the different regions of the colon, a representative microbial community is established in the three colon compartments, which differs both in composition and functionality in the different colon regions.

Further, porcine mucin is included in the reactors simulating the colon to take into account the colonisation of the mucous layer. Thus, the M-TripleSHIME™ permits culturing both the luminal and mucous-associated microbial community over periods of several weeks.

The M-TripleSHIME™ is run in four stages:
1. Stabilisation: After inoculation of the reactors with a fresh faecal sample taken from a healthy adult, a two-week stabilisation period allows the microbial community to differentiate in the different reactors depending on the local environmental conditions. During this period, the basic nutritional matrix is provided to support the maximum diversity of the gut microbiota originally present in the faecal inoculum.
2. Control: During this two-week period, a standard nutrient matrix is dosed into the model for a period of 14 days. The baseline microbial community composition and activity in the different reactors is determined by analysis of samples and is used as a reference.
3. Treatment: The SHIME system is operated under normal conditions for 3 weeks, but with the standard nutrient matrix supplemented with the HMOs. The HMOs tested are 2′-FL, LNnT and a 4:1 mixture of 2′-FL and LNnT.
4. Washout: During this two-week period, the SHIME system is again run with the standard nutrient matrix only.

Sample of the liquids in each reactor are collected regularly and are analysed for microbial metabolites and the composition of the resident microbial community using 16S rRNA sequencing.

The results from the fermentation system show that HMOs impact the base-acid consumption meaning that HMOs are fermented both in the proximal colon and, to a lesser extent the distal colon. The bacterial metabolite analysis show that HMO treatment induce an immediate increase in total SCFA production in both colon regions, mainly due to increase in the production of acetate and propionate. During the third week of HMO treatment, butyrate is increased.

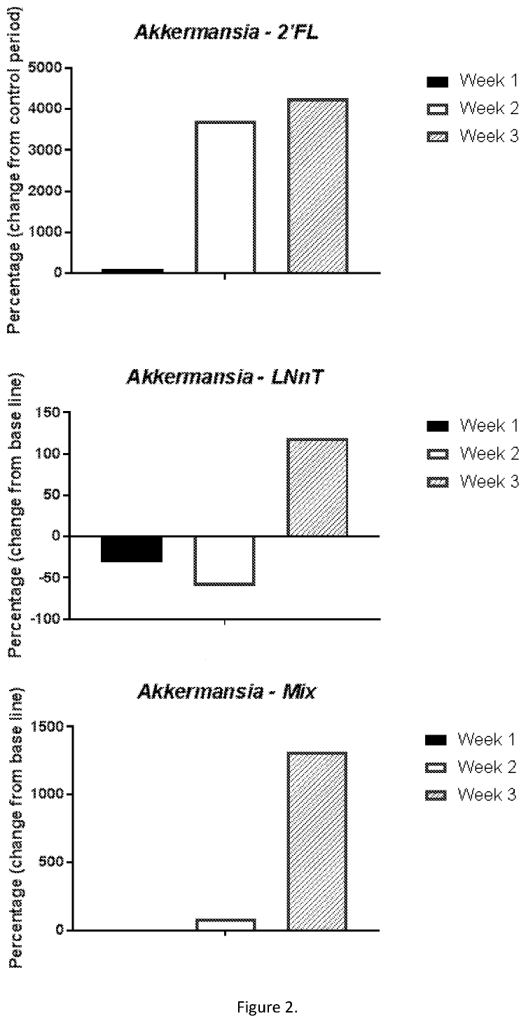

The profiling of the microbial community showed that during the 3 weeks treatment period the abundance of Akkermansia increased in the distal part of the colon regions. Practically no such change occurred in the proximal part. FIG. 2 shows the change in the Akkermansia abundance in the distal part during the treatment period (values are calculated as change in percent compared to the control period).

It could be seen that feeding the M-TripleSHIME™ with HMOs impacted the production of SCFA and treatment increased the abundance of Akkermansia in the distal part of the colon regions.

### Example 3

The HMOs 2′-FL and LNnT are introduced into a rotary blender in a 4:1 mass ratio. An amount of 0.25 mass % of magnesium stearate is introduced into the blender and the mixture blended for 10 minutes. The mixture is then agglomerated in a fluidised bed and filled into 5 gram stick packs and the packs sealed.

1.-3. (canceled)

4. A pack comprising at least 14 individual daily doses of an effective amount of at least one human milk oligosaccharide (HMO), and instructions for use in increasing the abundance of Akkermansia in the gastro-intestinal tract of a human.

5. The pack for the use according to claim 4, in which each daily dose contains 1 g to 15 g, preferably 2 g to 10 g of the at least one HMO.

6.-12. (canceled)

13. A method for increasing the abundance of Akkermansia in the gastro-intestinal tract of a human, the method comprising orally or enterally administering to the human an effective amount of a human milk oligosaccharide (HMO).

14. The method according to claim 13, in which the abundance of Akkermansia is increased in the mucosal layer of the gastro-intestinal track.

15. The method according to claim 14, in which the abundance of Akkermansia is increased in the colon.

16. The method according to claim 13, in which the abundance of Bifidobacterium is also increased.

17. The method according to claim 13, wherein the human suffers from an enteropathogenic infection.

18. The method according to claim 13, wherein the human suffers from type 2 diabetes, obesity and/or a liver disease.

19. The method according to claim 18, in which the amount administered is sufficient to improve intestinal permeability and/or increase insulin sensitivity.

20. The method according to any of claim 13, wherein the human suffers from inflammation related to gastro-intestinal condition.

21. (canceled)

22. The method according to claim 13, wherein the human suffers from a gut-brain disorder.

23. (canceled)

24. The method according to claim 13, wherein the human suffers from a food intolerance and/or sensitivity.

25. (canceled)

26. The method according to claim 13, wherein the human suffers from an impaired gut barrier function.

27. The method according to claim 13, wherein the human exhibits autism like behaviour.

28. (canceled)

29. The method according to claim 13, in which the human is administered an amount of 1 g to 15 g of the at least one HMO per day.

30. The method according to claim 13, wherein the HMO is administered to the human for at least 14 days.

31. The method according to claim 13, wherein the HMO comprises 2′-FL, 3-FL, DFL, LNT, LNnT, 3′-SL, 6′-SL, LNFP-I or a mixture thereof.

32. (canceled)

33. The method according to claim 13, wherein the HMO is a mixture of a fucosylated HMO and a non-fucosylated HMO.

34. The method according to claim 33, wherein the mixture comprises at least one of 2′-FL and DFL, and at least one of LNnT and LNT.

35. The method according to claim 34, wherein the mixture comprises 2′ FL and LNnT.

36. The method according to claim 13, comprising enterally administering to the human:
in a first step for a period of at least 7 days a first amount of a human milk oligosaccharide, or a synthetic composition comprising a first amount of a human milk oligosaccharide, wherein the first amount is effective to increase the abundance of Akkermansia in the gastro-intestinal tract of the human, and
in a second step for an additional period of least 7 days, a second amount of a human milk oligosaccharide, or a synthetic composition comprising a second amount of a human milk oligosaccharide, wherein the second amount is effective to maintain the abundance of Akkermansia in the gastro-intestinal tract of the human.
